(19) United States
(12) Patent Application Publication
(10) Pub. No.: US 2014/0170737 A1
(43) Pub. Date: Jun. 19, 2014

(54) STACKABLE MICRO-FLUIDIC CELLS

(71) Applicants: Pierre Indermuhle, Berkeley, CA (US); Alexander Stuck, Wettingen (CH); Alicia Hegglin, Zug (CH)

(72) Inventors: Pierre Indermuhle, Berkeley, CA (US); Alexander Stuck, Wettingen (CH); Alicia Hegglin, Zug (CH)

(21) Appl. No.: 14/187,231

(22) Filed: Feb. 22, 2014

Related U.S. Application Data
(63) Continuation-in-part of application No. 13/195,918, filed on Aug. 2, 2011.
Application No. 13/195,918 is a continuation-in-part of application No. 13/195,922, filed on Aug. 2, 2011, now Pat. No. 8,697,005.

(51) Int. Cl. G01N 33/53

(52) U.S. Cl. CPC G01N 33/5304
USPC 435/287.2

(57) ABSTRACT

An assay assembly that includes an assay bar having a plurality flow cells is disclosed. Each of the flow cells includes an inlet, an outlet, and an inside surface defining an inner volume. The outlet includes a valve that is configured to retain liquid within the inner volume of the flow cell. Each assay bar is configured to be reversibly stacked upon another assay bar, such that the flow cells of the stacked assay bars are in fluid communication with each other. This way, the outlet of a first flow cell of a first assay bar is in fluid communication with the inlet of a second flow cell of a second assay bar. The assay assembly may include a multitude of assay bars to form a composite assay assembly, with the flow cells of the stacked assay bars being in fluid communication with each other.

### CROSS-REFERENCE TO RELATED APPLICATIONS

This application is a continuation-in-part application of U.S. patent application Ser. No. 13/195,918, filed on Aug. 2, 2011, and is also a continuation-in-part application of U.S. patent application Ser. No. 13/195,922, filed on Aug. 2, 2011.

### FIELD OF THE INVENTION

The field of the present invention relates to biochemical assays and, more particularly, to stackable micro-fluidic cells and related assemblies that may be used in biochemical assays.

### BACKGROUND OF THE INVENTION

The high cost of certain biochemical and chemical reagents often requires scientists to use such reagents in a parsimonious manner. However, consuming only small amounts of reagents does not always translate into the desired amount of cost savings. As most laboratories have realized, handling very small amounts of reagents will often create increased labor costs (particularly if the assay is performed manually), and/or it may otherwise require expensive capital equipment to accommodate the handling of small volumes. Accordingly, there is a continued and growing demand for a technology and assay platform that not only conserves the use of expensive reagents, but also provides a user-friendly and cost-effective approach to performing analytical assays from a labor perspective. Such technology and assay platform may be useful in, for example, the multiplexed dispensing of reagents during the performance of certain binding assays, e.g., an ELISA (enzyme-linked immunosorbent assay).

As the following will demonstrate, the subject invention addresses the foregoing demands and many others.

### SUMMARY OF THE INVENTION

According to certain aspects of the present invention, an assay assembly that comprises at least one assay bar is provided. The invention provides that each assay bar includes a plurality flow cells, with each flow cell preferably being configured as a capillary tube. In addition, the invention provides that each of the flow cells will include an inlet, an outlet, and an inside surface defining an inner volume. The outlet will preferably comprise a valve that is configured to retain liquid within the inner volume of the flow cell. As described further below, the valve of each flow cell will preferably consist of a capillary barrier or a passive valve.

According to certain preferred aspects of the invention, each assay bar is configured to be reversibly stacked upon another assay bar, such that the flow cells of the stacked assay bars are in fluid communication with each other. This way, the outlet of a first flow cell of a first assay bar is in fluid communication with the inlet of a second flow cell of a second assay bar. The assay assembly may include a multitude of assay bars to form a composite assay assembly, with the flow cells of the stacked assay bars being in fluid communication with each other. When a first assay bar is stacked upon a second assay bar (or a multitude of assay bars are stacked upon each other), a composite assembly is created, with the resulting composite flow cells having a single inlet (at the top of the assembly), a single outlet (at the bottom of the assembly), and a composite inner volume. According to such embodiments, the composite assembly is configured to receive the liquid at the single inlet and to retain the liquid within the composite inner volume. Still further, and following the provision of liquid to the composite inner volume of the assembly, the invention provides that the composite assembly will be configured to allow the assay bars to then be unstacked and split into individual assay bars, while retaining the liquid within the inner volumes of the flow cells of the constituent assay bars.

The invention provides that the inner volume of each flow cell will preferably exhibit a cylindrical (or approximately cylindrical) dimension. According to certain aspects of the invention, the inlet and the outlet of the flow cells may, optionally, each comprise a mating element. In such embodiments, a first mating element of an outlet is configured to receive (or be inserted into) a corresponding second mating element of the inlet of another flow cell—thereby creating a more secure connection between such flow cells of separate assay bars, when the assay bars are stacked upon each other as described herein. Still further, according to certain aspects of the invention, the inlet, the outlet, and/or inside surface of each flow cell may, optionally, be provided with a coating that is effective to assist in retaining liquid within the flow cell.

According to yet additional aspects of the invention, the outlet of each flow cell may protrude from a bottom surface of the assay bar, and the inlet of each flow cell may protrude from an upper surface of the assay bar. In certain preferred embodiments, the invention provides that the flow cells will be grouped into clusters, and preferably arranged in a two-dimensional matrix. The invention provides that the assay bars described herein may comprise a single row of flow cells (or a row of flow cell clusters). Alternatively, the assay bars may exhibit the dimensions of an assay plate, e.g., each assay bar may comprise at least two rows and at least two columns of flow cells—or, in some cases, the assay bar may exhibit the dimensions of a conventional 96-well plate, albeit including flow cells instead of wells (or clusters of flow cells), as described further below.

The above-mentioned and additional features of the present invention are further illustrated in the Detailed Description contained herein.

### BRIEF DESCRIPTION OF THE FIGURES

FIG. 1: (A) Perspective and magnified view of an assay bar, showing the clusters of flow cells included therein. (B) Perspective view of the assay bar of (A) above, showing a total of eight clusters of four flow cells. These views further show the inlets of the flow cells protruding from the top surface of the assay bar.

FIG. 2: A side cross-sectional view of the flow cells described herein, which further shows the position of the valves that are configured to stop and retain liquid within the flow cells. (A) and (B) represent two different valve configurations. The valve of configuration (A) includes a narrowing of the flow cell above the valve, and an expansion of the flow cell below the valve. The valve of configuration (B) only includes a narrowing of the flow cell above the valve.

FIG. 3: (A) Perspective view of two assay bars (having eight clusters of four flow cells) stacked on top of each other. The gap between the two assay bars is created by protruding inlets and outlets of the flow cells. (B) Perspective and transparent view of the two assay bars of (A) above.

FIG. 4: A side cross-sectional view of two flow cells of a first assay bar, stacked upon two flow cells of a second assay bar, with the location of the valves also being shown (along with the two valve configurations, (A) and (B), described in FIG. 2 above).

FIG. 5: (A) Perspective views of flow cells having the same dimensions in separate configuration. (B) Perspective views of the same flow cells in (A) stacked on top of each other to form a composite flow cell.

FIG. 6: (A) Perspective views of two flow cells of different dimensions in separate configuration. (B) Perspective views of the same flow cells in (A) stacked on top of each other to form a composite flow cell.

FIG. 7: Perspective views of two flow cells with mating elements at the inlet and outlet portions thereof.

FIG. 8: (A)-(D): Side cross-sectional views of flow cells in individual mode, showing capillary forces causing the liquid to move into the inner volume of the flow cell when the liquid comes into contact with the inlet (with the liquid stopping at the outlet of the flow cell). (E)-(I): Side cross-sectional views of two sets of flow cells stacked upon each other and configured to receive liquid according to the same mechanism in (A)-(D) above, i.e., to fill a composite flow cell.

### DETAILED DESCRIPTION OF THE INVENTION

The following will describe, in detail, several preferred embodiments of the present invention. These embodiments are provided by way of explanation only, and thus, should not unduly restrict the scope of the invention. In fact, those of ordinary skill in the art will appreciate upon reading the present specification and viewing the present drawings that the invention teaches many variations and modifications, and that numerous variations of the invention may be employed, used and made without departing from the scope and spirit of the invention.

The technology and assay platform provided by the present invention is particularly useful in the multiplexed dispensing of reagents during the performance of certain binding assays, e.g., an ELISA (enzyme-linked immunosorbent assay). The goal of an ELISA (enzyme-linked immunosorbent assay) is to detect certain target molecules of interest within a liquid. This detection step is enabled through an antibody-antigen (or Fab fragment-antigen) reaction which takes place at the surface of, for example, a plastic well or on the surface of beads included within the assay reagents. The standard formats used today for such assays include 96-well plates, 12-well strips, 8-well strips, and 384-well plates (with many laboratories often converting from a 96-well plate (200 μl/well) format to a 384-well plate (50 μl/well) format to save costs, conserve reagents, and/or to increase assay throughput).

A standard ELISA protocol consists of the following steps (the amount of additional modifications or reduction of certain steps depends on the specific ELISA assay being performed). First, the surface of the plate is prepared and a blocking agent is applied (and allowed to incubate for some time, before being removed and washed with an appropriate buffer solution). Next, an anchoring molecule, such as an antibody, is applied and allowed to bind to the plate surface (which is followed by a buffer washing step to remove unbound anchoring molecules). The sample is next applied to the plate (along with a control sample being added to its own dedicated wells). The antigen (target molecule) that is contained within the sample (and the control in separate wells) will then react and bind with the anchoring molecule. The plate is then washed, followed by the application of a detection molecule, which will allow the amount of antigen bound (and therefore contained within the sample) to be detected and quantitated using standard laboratory equipment (e.g., a standard multi-well optical reader). In many cases, the detection molecule represents another antibody that is capable of binding to the target antigen, with the antibody being labeled with an enzyme or fluorescent tag (which an optical reader may detect).

Since the 96-well format is currently used as the standard assay format, each step of an ELISA protocol will often consume a reagent volume of 100-200 μl. Over the life of an ELISA assay, the cumulative reagent consumption is often a few milliliters per reaction (per well), which can translate into fairly significant costs. The invention provides that it would be desirable to provide a technology and new assay platform that allows laboratories to continue to use the types of established ELISA protocols (and existing equipment) described above, while simultaneously reducing the amount of reagent volume that is required to carry out such protocols (which will thereby reduce the total cost of the assay). In addition, it would be desirable to reduce the number of pipetting steps (and therefore labor) that is required to perform these assays.

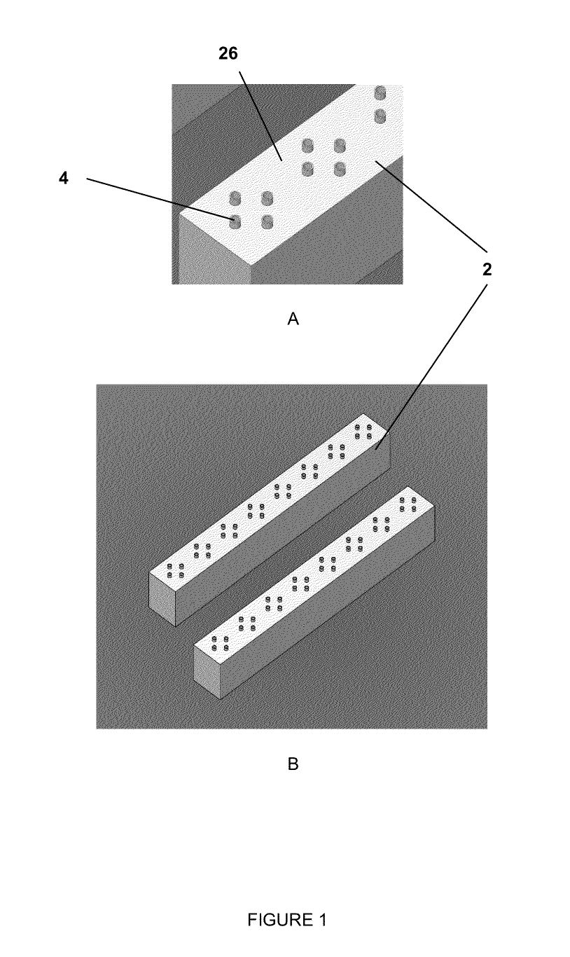

Referring now to FIGS. 1-8, according to certain preferred embodiments of the present invention, an assay assembly that comprises at least one assay bar 2 is provided. The invention provides that each assay bar 2 includes a plurality flow cells 4, with each flow cell 4 preferably being configured as a capillary tube. In addition, the invention provides that each of the flow cells 4 will include an inlet 6, an outlet 8, and an inside surface 10 defining an inner volume 12. The dimensions of the inlet 6 and outlet 8 of each flow cell 4 are preferably the same among the flow cells 4 of each assay bar 2. The invention further provides that the outlet 8 will preferably comprise a valve 14 that is configured to retain liquid within the inner volume 12 of the flow cell 4.

In certain preferred embodiments, the assay bar 2 will consist of a plastic bar, e.g., comprised of polypropylene or polystyrene, which contains eight symmetrically arranged flow cells 4 (or eight clusters of flow cells 4, with each cluster having two-to-twelve separate flow cells 4). The assay bars 2 and flow cells 4 may be manufactured through, for example, plastic extrusion or injection molding. In other embodiments, the assay bar 2 (and/or the flow cells 4 thereof) may be comprised of a glass material. In addition, the invention provides that the flow cells 4 may be manufactured separate and apart from the remaining portions of the assay bar 2 (matrix); and then inserted into the assay bar 2, e.g., the flow cells 4 may be inserted into certain holes of an assay bar 2. Still further, the flow cells 4 may be created by drilling the flow cells 4 into an existing assay bar 2/matrix.

The invention provides that the flow cells 4 are open at both ends (the inlet 6 and outlet 8), such that liquid can freely enter (or be forced to enter) and be soaked and retained within the inner volume 12 of each flow cell 4. As such, an ELISA assay may be performed within the inner volume 12 of each flow cell 4. In addition, the invention provides that when the flow cells 4 are embedded within the assay bar 2 in clusters, each cluster will preferably exhibit the same pattern of flow cells 4 (so that the flow cells 4 of different assay bars 2 may be aligned and stacked upon each other as described herein).

As mentioned above, the flow cells 4 will preferably be configured as capillary tubes. At small diameters, i.e., in the range of 1 millimeter and below, surface tension dominates liquid behavior on surfaces and within contained volumes. Capillary forces will pull a liquid inside a capillary having a small inner diameter and a hydrophilic surface—and will prevent the liquid from flowing out (i.e., due to the presence of a capillary barrier). However, if the capillary barrier is broken, e.g., by bringing a second capillary into fluidic contact with a first capillary, the liquid will start flowing into the second capillary. The flow will stop when the capillaries are separated or when the second capillary is full. Similarly, a stack of flow cells 4 described herein, which exhibit the appropriate dimensions and surface properties, can be filled with a liquid through the inlet 6 of the flow cell 4. The liquid will fill the inner volume 12 of the flow cells 4 and stop at the outlet 8 of the flow cell 4 (due to capillary forces, which represent a type of valve 14 that may be used with the invention). When the assay bars 2 are stacked upon each other, as described herein, the invention provides that the flow cells 4 only need to be closely assembled to prevent the liquid from leaking at their junctions.

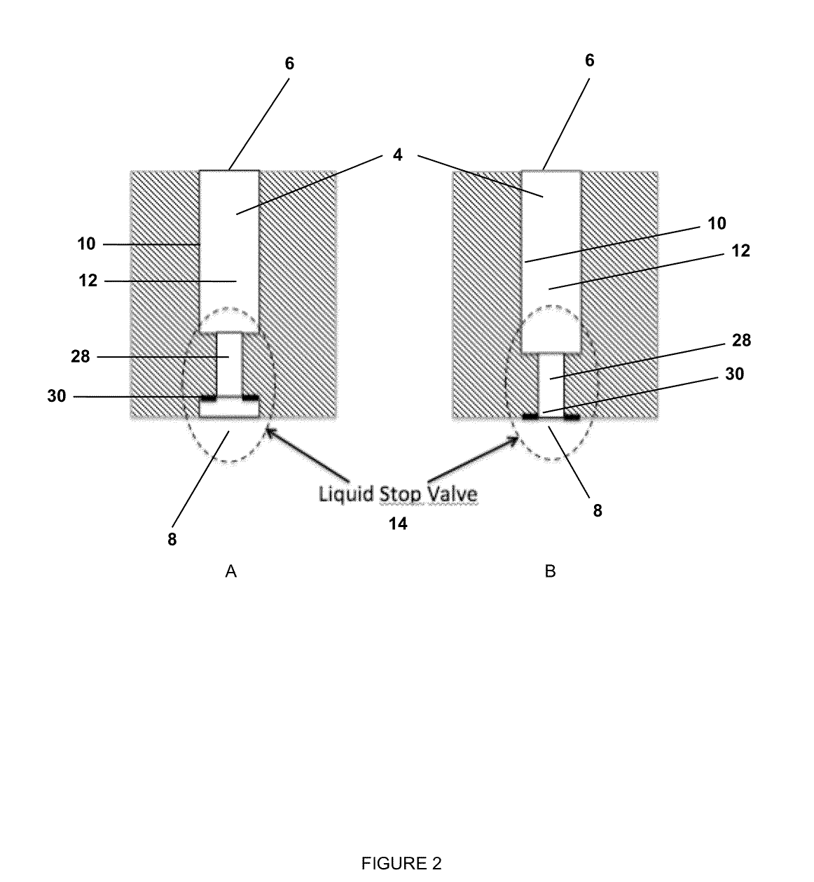

Referring now to FIG. 2, the valve 14 may be configured to exhibit a narrower section 28 that expands abruptly at one end, following the valve 14 (FIG. 2(A)). The invention provides that the expanded region may be rectangular in dimension, but could also be tapered or curved. In addition, FIG. 2 shows the regions 30 of the valve 14 that may, optionally, exhibit a modified surface tension relative to the other parts of the flow cell 4 (i.e., the regions 30 are indicated by a thicker line at the bottom of the valve 14). The invention provides that surface modification can be achieved by different means. For example, the invention provides that chemical coatings (or a local plasma treatment) may be applied to such regions 30 of the valve 14. In addition, the invention provides that micro- or nano-structuring of the region 30 may be employed using, for example, the well-known Lotus effect to achieve non-wetting conditions in such regions 30. The invention provides that changing the surface tension at the end of the valve 14 in this manner will serve to increase the pressure drop across the valve 14 (and, furthermore, to increase the amount of force required to cause liquid to exit the flow cell 4).

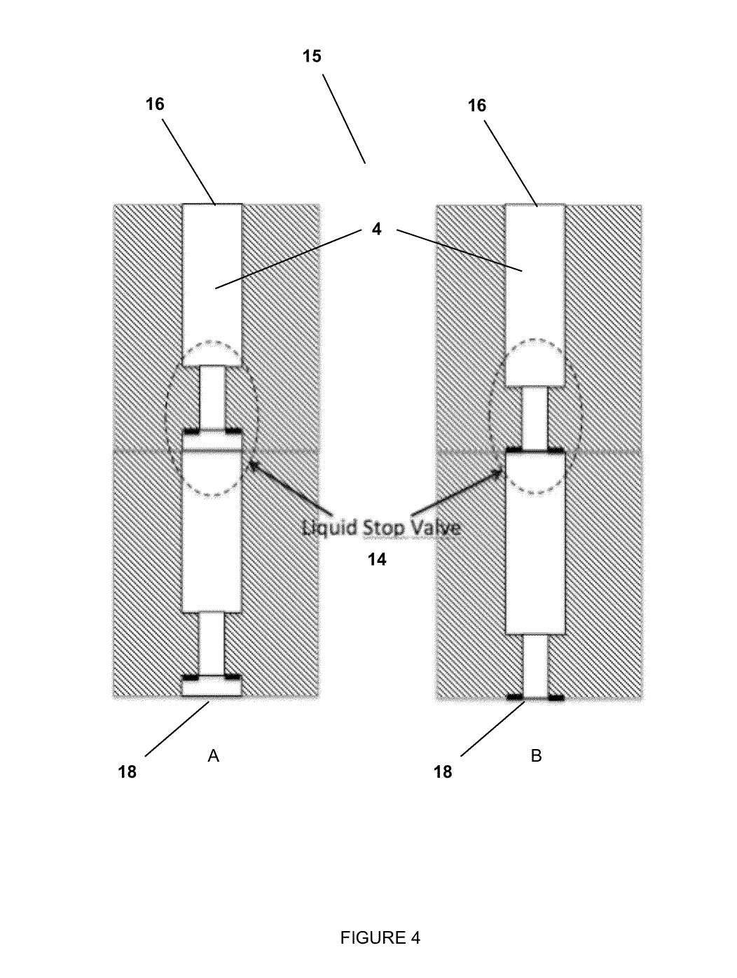

Referring to FIGS. 2 and 4, the valve 14 of each flow cell 4 will preferably consist of a capillary barrier or a passive valve, with the inner volume 12 of each flow cell 4 preferably exhibiting a cylindrical (or approximately cylindrical) dimension. The invention provides that if the flow cells 4 are non-wetting (i.e., the flow cells 4 do not exhibit a hydrophilic inside surface 10), liquid may be pressed into the flow cells 4, e.g., via a syringe or pneumatically pulled through the flow cells 4 with a pump applied to the bottom surface of the assay bar 2. Alternatively, if the flow cells 4 have been wetted by a liquid, passive liquid stop valves 14 near the outlet 8 of the flow cell 4 will prevent the liquid from flowing out. As explained above, the invention provides that the stop valves 14 can be made by locally modifying the surface properties of inside surface 10 of the flow cell 4 from hydrophilic to hydrophobic—and/or by creating an abrupt expansion of the flow cell 4 (FIG. 2(A)). In such embodiments, the invention provides that the liquid will not enter the hydrophobic region and/or expanded region due to prevailing capillary forces. In certain embodiments, the outermost portion of the valve 14 may further comprise an edge or lip, which may further serve to retain liquid within the flow cell 4.

The invention provides that the valve 14 will be opened if the liquid pressure at the liquid meniscus overcomes the barrier created by the valve 14, e.g., the capillary forces that are present at the location of the valve 14. In certain embodiments, the invention provides that additional pressure can be applied through a syringe, which actively fills the capillary tubes of the flow cells 4. Alternatively, the invention also provides that the lowering of gas pressure through a pump (or other pneumatic device) applied to the bottom of the assay bar 2 may overcome the capillary forces at the stop valve 14. Still further, the invention provides that the valve 14 may also be opened by applying a mechanical wave to the assay bar 2, which agitates the liquid through the stop valve 14. Depending on the valve 14 configuration, the agitation of the assay bar 2 can be low or high frequency. In such embodiments, once the liquid has passed through the valve 14, and the valve 14 surface has been covered by the liquid, the surface tension at the valve 14 will not exert a net force on the liquid, such that the liquid can freely flow through the valve 14 to a neighboring flow cell 4 (when the flow cells 4 of multiple assay bars 2 are stacked upon each other).

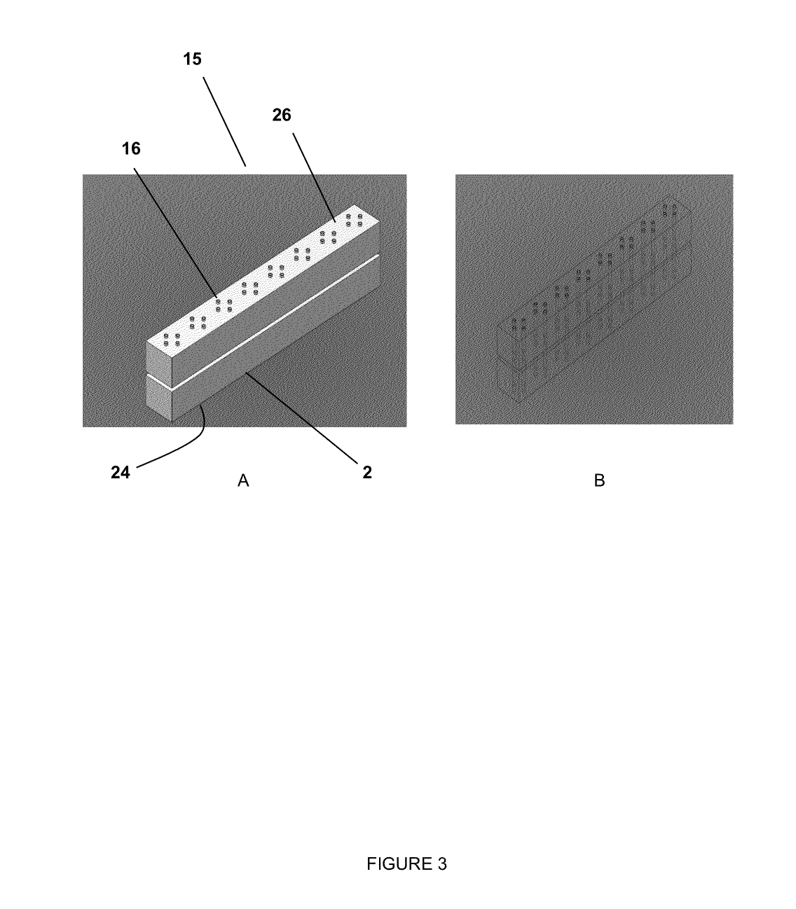

Referring to FIGS. 3 and 4, according to certain preferred embodiments of the invention, each assay bar 2 is configured to be reversibly stacked upon another assay bar 2, such that the flow cells 4 of the stacked assay bars 2 are in fluid communication with each other. This way, the outlet 8 of a first flow cell 4 of a first assay bar 2 is in fluid communication with the inlet 6 of a second flow cell 4 of a second assay bar 2. The assay assembly may include a multitude of assay bars 2 to form a composite assay assembly (e.g., 2, 3, 4, 5, or more stacked assay bars 2), with the flow cells 4 of the stacked assay bars 2 being in fluid communication with each other. When a first assay bar 2 is stacked upon a second assay bar 2 (or a multitude of assay bars 2 are stacked upon each other), a composite assembly 15 is created, with the resulting composite flow cells 4 having a single inlet 16 (at the top of the assembly 15), a single outlet 18 (at the bottom of the assembly 15), and a composite inner volume between the inlet 16 and outlet 18. According to such embodiments, the composite assembly 15 is configured to receive the liquid at the single inlet 16 and to retain the liquid within the composite inner volume.

More particularly, the invention provides that the inlet 6 of the first flow cell 4 of the assembly 15 acts as the inlet 16 of the composite flow cell and the outlet 8 of the last (bottommost) flow cell 4 of the assembly 15 works as the outlet 18 of the composite flow cell. The inner volume of a composite flow cell is the resulting volume of the inner volumes of its constituent flow cells 4. The invention provides that liquid can be dispensed into the composite flow cell through its inlet 16—and the liquid may be held within the inner volume of the composite flow cell, if so desired, by closing the valve 14 at its outlet 18. The invention provides that such liquid may then be released through the outlet 18 of the composite flow cell by opening the valve 14 or otherwise breaking the capillary barrier at such location (outlet 18).

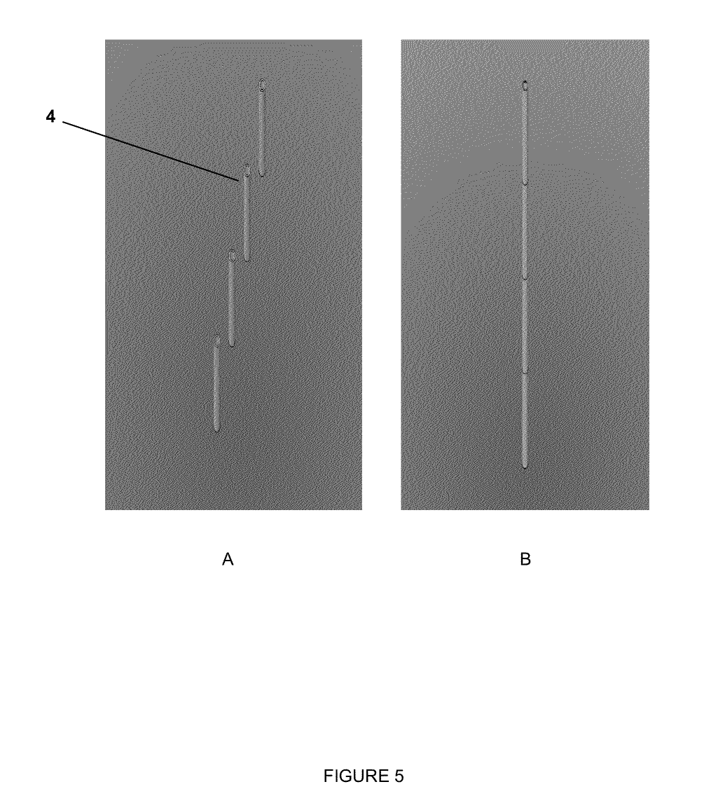

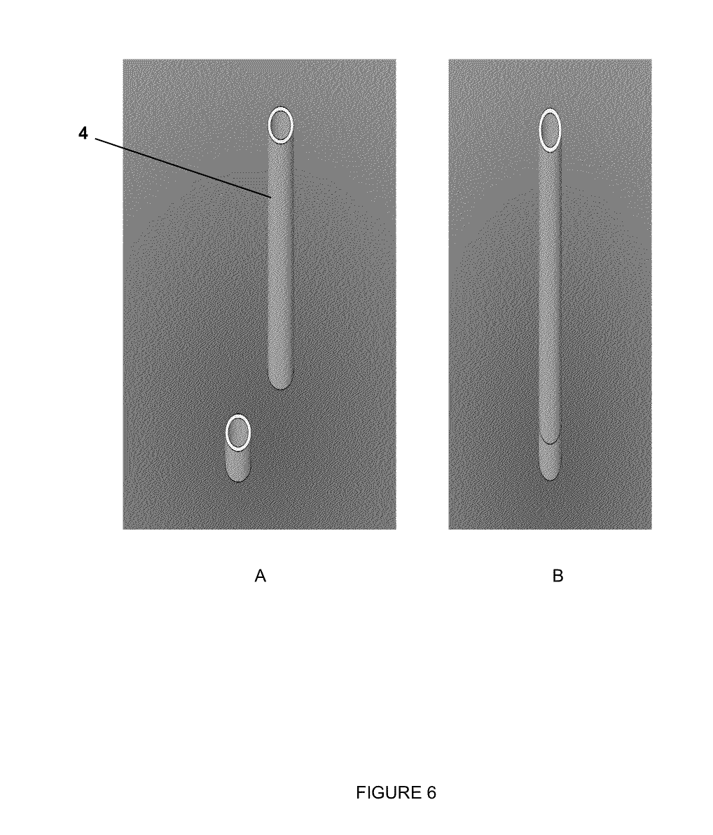

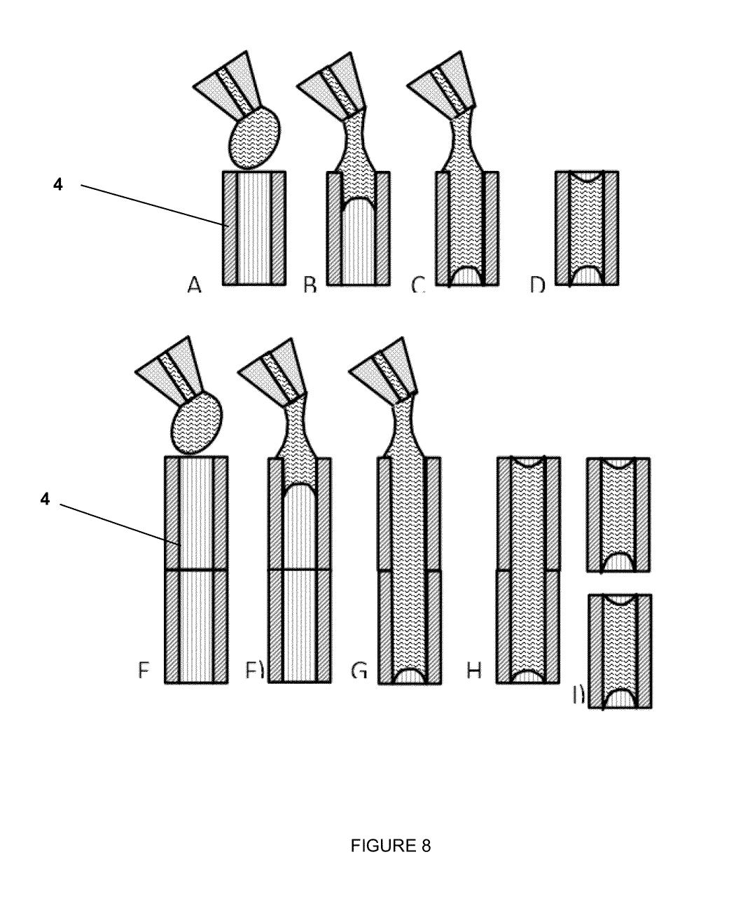

Still further, the invention provides that the composite assembly 15 will be configured to allow the assay bars 2 to be unstacked and split into individual assay bars 2 (FIG. 8), while retaining the liquid within the inner volume of the flow cells 4 of each assay bar 2. The invention provides that only when all flow cells 4 have been correctly stacked and aligned on each other, will liquid be allowed to travel through the composite inner volume and be prevented from exiting the outlet 18. In addition, the invention provides that in certain embodiments, all the flow cells 4 will have the same inner volume (FIG. 5); whereas, in other embodiments, the flow cells 4 may have different inner volumes (FIG. 6).

When the assay bars 2 described herein are placed adjacent to each other in the traditional ELISA configuration, i.e., not stacked on top of each other, the molecules which can be detected by an optical reader are actually bound to the inside surface 10 of the flow cells 4. As such, the detected molecules are oriented perpendicular to the traditional well floors of a standard ELISA plate. The invention provides that this perpendicular orientation is effective to increase the optical detection sensitivity provided by the assay bar 2, vis-à-vis a longer light path for the absorption of the illuminating light of the optical reader.

In addition, the invention provides that the volume-to-surface ratio of the reagents in the flow cell 4 is geometrically increased compared to normal ELISA plates. This also contributes to an increase in measurement sensitivity. Still further, in certain embodiments the bottom surface of the assay bar 4 (around each flow cell 4) may be curved, which may operate as a collective lens to focus the light from the measurement system (optical reader) on the flow cell 4 walls, which will also increase the measurement sensitivity. In addition, the invention provides that a similar effect can be achieved using suitable diffractive optical structures, e.g., holograms placed at the bottom interface of each flow cell 4. In such embodiments, the invention provides that a refractive or diffractive lens (or micro-lens) arrangement may be employed around each flow cell 4 to not only focus the light onto the flow cell 4 walls, but also to collect and direct the light emitted from the walls towards the measurement system (optical reader) and, therefore, increase the measurement sensitivity of the assay.

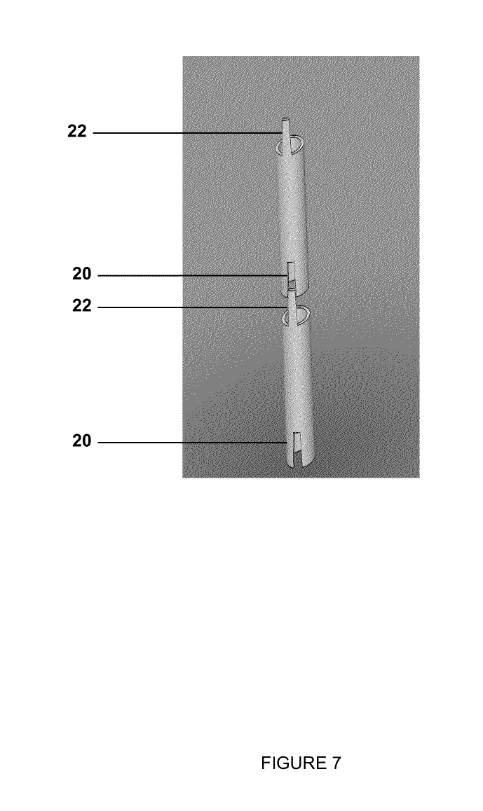

Referring to FIG. 7, according to certain embodiments of the invention, the inlet 6 and the outlet 8 of the flow cells 4 may, optionally, each comprise a mating element. For example, in such embodiments, a first mating element 20 of an outlet 8 (e.g., a region 20 that represents a notch or is cut out of the perimeter of the outlet 20) is configured to receive a corresponding second mating element 22 of the inlet 6 (e.g., a protrusion 22 that is configured to be fittingly inserted into the region 20) of another flow cell 4, thereby creating a more secure connection between such flow cells 4 of separate assay bars 2, when the assay bars 2 are stacked upon each other as described herein.

According to yet additional embodiments of the invention, the outlet 8 of each flow cell 4 may protrude from a bottom surface 24 of the assay bar 2, and the inlet 6 of each flow cell 4 may protrude from an upper surface 26 of the assay bar 2 (FIG. 1). The inlet 6 and the outlet 8 of the flow cell 4 may protrude from the local surfaces (or matrix) of the assay bar 2 in this manner to provide enhanced fluidic control. For example, the invention provides that a protruding inlet 6 allows for an easier filling of the flow cell 4. In addition, protruding inlets 6 and outlets 8 prevent liquid from wicking between flow cells 4 or between assay bars 2 when they are stacked upon each other.

As mentioned above, in certain preferred embodiments, the invention provides that the flow cells 4 will be grouped into clusters, and preferably arranged in a two-dimensional matrix (FIG. 1(B)). The invention provides that the assay bars 2 described herein may comprise a single row of flow cells 4 (or clusters of flow cells 4). Alternatively, the assay bars 2 may exhibit the dimensions of an assay plate, e.g., each assay bar 2 may comprise at least two rows and at least two columns of flow cells 4 (or clusters of flow cells 4). In other embodiments, the assay bars 2 may exhibit the dimensions of a standard 96-well assay plate, e.g., each assay bar 2 may comprise at least eight rows and twelve columns of flow cells 4 (or clusters of flow cells 4).

The many aspects and benefits of the invention are apparent from the detailed description, and thus, it is intended for the following claims to cover all such aspects and benefits of the invention that fall within the scope and spirit of the invention. In addition, because numerous modifications and variations will be obvious and readily occur to those skilled in the art, the claims should not be construed to limit the invention to the exact construction and operation illustrated and described herein. Accordingly, all suitable modifications and equivalents should be understood to fall within the scope of the invention as claimed herein.

What is claimed is:

1. An assay assembly comprising an assay bar that includes a plurality flow cells, wherein each of the flow cells comprises an inlet, an outlet, and an inside surface defining an inner volume, wherein said outlet comprises a valve that is configured to retain liquid within the inner volume of the flow cell, wherein the assay bar is configured to be reversibly stacked upon another assay bar, such that the flow cells of the stacked assay bars are in fluid communication with each other, such that the outlet of a first flow cell of a first assay bar is in fluid communication with the inlet of a second flow cell of a second assay bar.

2. The assay assembly of claim 1, wherein the valve consists of a capillary barrier or a passive valve.

3. The assay assembly of claim 1, wherein the inner volume is cylindrical or approximately cylindrical.

4. The assay assembly of claim 1, wherein the inlet and the outlet of the flow cells each comprise a mating element, wherein a first mating element of an outlet is configured to receive or be inserted into a corresponding second mating element of the inlet of another flow cell.

5. The assay assembly of claim 4, wherein the inlet, the outlet, the inside surface, or combinations thereof comprise a coating that is effective to assist in retaining liquid within the flow cell.

6. The assay assembly of claim 1, wherein the outlet protrudes from a bottom surface of the assay bar.

7. The assay assembly of claim 1, wherein the inlet protrudes from an upper surface of the assay bar.

8. The assay assembly of claim 1, wherein the flow cells are grouped in clusters.

9. The assay assembly of claim 8, wherein the clusters are arranged in a two dimensional matrix.

10. The assay assembly of claim 1, wherein the assay bar is a plate that comprises at least two rows and at least two columns of flow cells.

11. An assay assembly comprising two or more assay bars that each include a plurality flow cells, wherein each of the flow cells comprises an inlet, an outlet, and an inside surface defining an inner volume, wherein said outlet comprises a valve that is configured to retain liquid within the inner volume of the flow cell, wherein the assay bars are configured to be reversibly stacked upon each other, such that the flow cells of the stacked assay bars are in fluid communication with each other, wherein:
(a) when a first assay bar is stacked upon a second assay bar a composite assembly is created, with a composite flow cell having a single inlet, a single outlet, and a composite inner volume;
(b) the composite assembly is configured to receive the liquid at the single inlet and to retain the liquid within the composite inner volume; and
(c) the composite assembly is further configured to allow the assay bars to be unstacked and split into individual assay bars, while retaining the liquid within the inner volume of the flow cells of each assay bar.

12. The assay assembly of claim 11, wherein the inlet, the outlet, the inside surface, or combinations thereof comprise a coating that is effective to assist in retaining liquid within the flow cell.

13. The assay assembly of claim 11, wherein the valve consists of a capillary barrier or a passive valve.

14. The assay assembly of claim 11, wherein the outlet protrudes from a bottom surface of the assay bar.

15. The assay assembly of claim 11, wherein the inlet protrudes from an upper surface of the assay bar.

16. The assay assembly of claim 11, wherein the flow cells are grouped in clusters.

17. The assay assembly of claim 16, wherein the clusters are arranged in a two dimensional matrix.

18. The assay assembly of claim 11, wherein the assay bars are plates that comprise at least two rows and at least two columns of flow cells.
